(19) United States
(12) Patent Application Publication
(10) Pub. No.: US 2012/0289681 A1
(43) Pub. Date: Nov. 15, 2012

(54) CONJUGATES OF CELL-PENETRATING PEPTIDES AND PHOSPHORESCENT METALLOPORPHYRINS FOR INTRACELLULAR OXYGEN MEASUREMENT

(75) Inventors: Dmitri Papkovsky, Blarney (IE); Ruslan Dmitriev, Cork (IE)

(73) Assignee: UNIVERSITY COLLEGE CORK, NATIONAL UNIVERSITY OF IRELAND CORK, Cork (IE)

(21) Appl. No.: 13/143,309

(22) PCT Filed: Jun. 8, 2009
(86) PCT No.: PCT/IE09/00033

§ 371 Date: Sep. 27, 2011

(51) Int. Cl. C07K 7/08; C07K 7/06; C07K 7/02; C07K 2/00

(52) U.S. Cl. 530/327; 530/300; 530/328; 530/332

(57) ABSTRACT

A phosphorescent compound of general Formula I,

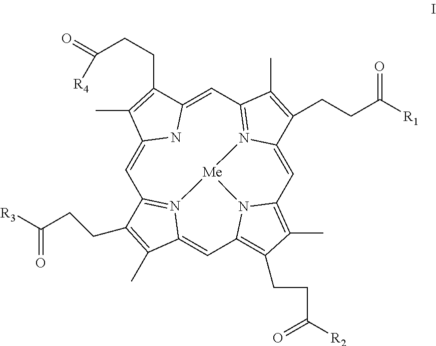

or phosphorescent analogs thereof, wherein:
   at least one of R1 to R4 has a formula X—Y, wherein Y is a peptide sequence providing cell penetration, and X is absent or is a chemical linker;
   the or each of the remaining R1 to R4 groups are, independently, lipophilic, uncharged chemical groups; and
   Me is selected from Pt²⁺ or Pd²⁺,
      which probe is capable of measurement of molecular oxygen within live respiring cells by quenched-phosphorescence detection.

### INTRODUCTION

The invention describes a family of phosphorescence compounds. In particular, the invention relates to the use of such phosphorescent compounds in the analysis of the molecular oxygen (O₂) status of live cells.

### BACKGROUND TO THE INVENTION

Quantification of O₂ by luminescence quenching has a number of attractive features, including reversible, non-chemical and non-invasive nature of sensing of O₂. As a result, this methodology is now actively used in various industrial and biomedical applications where measurement of O₂ is required (Papkovsky D B. Methods Enzymol., 2004, 381: 715-735). In biological systems, O₂ is the key metabolite of aerobic cells which is consumed continuously to generate energy in the form of ATP. In eukaryotic cells, O₂ is consumed mainly in the mitochondria, where it acts as the terminal acceptor of the electron transport chain which fuels oxidative phosphorylation. The levels of O₂ within the cell and the rate of O₂ consumption serve as useful indicators of metabolic status, bioenergetics, mitochondrial function of the cells. Alterations in cellular O₂ levels and consumption are implicated in a number of (patho)physiological conditions, including neurological, cardiovascular, metabolic disorders, cancer, inflammation, etc. Furthermore, dysbalance between O₂ demand and supply to the cells leading to a state of hypoxia is known to trigger adaptive responses of the cell through a complex network of signalling pathways, transcriptional and metabolic changes.

Monitoring of cellular O₂ consumption and O₂ levels in live respiring cells and tissues is therefore important for many areas of biomedical and life sciences. A number of O₂-sensitive probes and measurement techniques have been developed for these purposes. Thus, Rumsey (Rumsey W L et al. Science, 1988, v. 241(4873): 1649-51), Dunphy (Dunphy, I, et al. Anal Biochem 2002, 310(2): 191-8), Vinogradov (Vinogradov S A et al. U.S. Pat. No. 5,837,865, 1998), Wilson (U.S. Pat. No. 6,395,555, 2002), Hynes (Hynes, J., et al. J Biomol Screening, 2003, 8(3): 264-72), Cao (Cao Y. et al. Analyst, 2004, 129(8): 745-50) described probes and techniques for the sensing and imaging of O₂ in systems containing live cells and tissue, and for the measurement of biological O₂ consumption. These techniques normally employ O₂-sensitive probes based on phosphorescent Pt- and Pd-porphyrins and some related structures, luminescence of which is quenched by O₂. Such probes were designed primarily for use extracellularly and/or in large biological samples such as live tissues and organs, they are essentially impermeable to the cells.

There is also growing evidence that O₂ gradients localised near and within the cell contribute to the development of pathological conditions in vivo and adaptive responses to hypoxia. There is therefore a need in probes and experimental methodologies for the measurement of local, intracellular and sub-cellular O₂. Unfortunately, the range of probes and techniques that allow such measurements is very limited, while the number of biological problems and experimental tasks which require these measurements increases steadily.

Clark-type O₂ microelectrodes for polarographic measurement of intracellular O₂ have been described, however they are invasive, consumptive, complex to use and not able to provide the level of detail achievable with fluorescence-based sensors/probes. Several optical probes and methodologies for sensing intracellular O₂ were described, which include polymeric nanoparticles impregnated with oxygen-sensitive dyes loaded into the cells by microprojectile delivery (Koo Y E et al. Anal Chem, 2004, v. 76(9): 2498-505), polymeric particles impregnated with a fluorescent dye and coated with a phospholipid shell (‘lipobeads’) loaded into macrophages by phagocytosis (Ji J. et al. Anal Chem., 2001, 73(15): 3521-7); microspheres doped with the dye introduced into large plant cells by microinjection (Schmalzlin E., et al., Biophys. J. 2005, 89(2): 1339); hydrophilic metalloporphyrin dye complexed with albumin microinjected into skeletal muscle fibres (Howlett R A, J Appl Physiol. 2007, 102(4):1456-61). However, these systems have limitations in their complexity, invasiveness, low efficiency of loading, uneven distribution of the probe inside the cell, uncontrolled compartmentation and aggregation, significant cyto- and phototoxicity.

Thus, particulate probes based on polymeric composites have relatively large size, biocompatibility, toxicity and stability issues. Their delivery into the cell and to specific locations within the cell is difficult, if at all possible. Loading by projectile delivery, endocytosis or micro- or nano-injection is technically complex, inefficient, and often causes irreparable damage to the cell. Random distribution of the relatively small number of particles within the cell may give a poor representation of intracellular oxygen distribution.

Molecular O₂ probes can potentially circumvent the limitations of particulate probes, however, the existing probes of this type also have limitations. Low molecular weight probes based on free O₂-sensitive dyes also suffer from hydrophobicity, accumulation in cell membranes or binding to DNA, thus resulting in high toxicity, photochemical damage, or leakage from the cell. Whereas probes comprising macromolecular conjugates of O₂-sensitive dyes are more difficult to synthesize, they often have complex chemical composition and variable photochemical and sensing properties.

One approach to the loading of cells with O₂ probes is to use special reagents which provide active transport of chemical and biological components from the extracellular medium into the cell. Standard approaches used for facilitating transport of chemicals into the cell include, for example, liposomal transfer, facilitated endocytosis and pinocytosis by the cells, viral transfection, etc. However, these methods require additional reagents, experimental steps and specialised equipment, they are highly dependent on the nature of chemical to be delivered into the cell, cell type, medium and other loading conditions. In many cases, cell loading by such techniques is rather low or even problematic for the cells, stressful and not very reproducible.

It is therefore evident, that one of the main problems with existing O₂ probes is in their delivery into the cell, which is often complex, inefficient, depends on the cell type, medium used or the presence of additional reagents/steps which provide facilitated transport of the probe into the cell. The use of such probes is associated with a significant impact on the cell and cellular function, due to the invasive nature of the probe and loading technique of the impact on the cell via chemical or photo-toxicity of the probe or loading method. Besides the general problem of delivery into the cell, targeting the O₂ probe to specific locations within the cell and conducting measurement of O₂ in these locations are even more problematic. All this limits the applicability of current O₂ probes and the information that these probes and measurement systems can potentially provide about (intra)cellular O₂.

### STATEMENTS OF INVENTION

The present invention is directed towards providing improved probes and methodologies for the measurement of (sub)cellular O₂. It addresses many of the problems with the current probes and techniques, and provides a new family of advanced O₂ probes, which are based on the derivatives of phosphorescent Pt- and Pd-porphyrin dyes and which are designed specifically for intracellular use and for sensing local O₂ gradients in live respiring cells and tissues.

According to the invention, there is provided a phosphorescent compound of general Formula I,

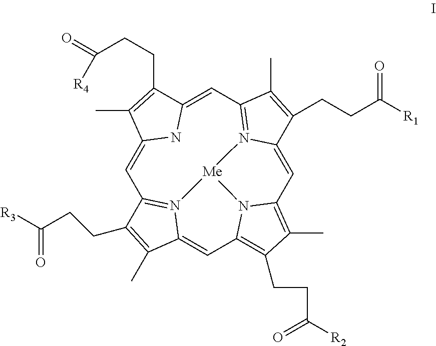

or phosphorescent analogs thereof, wherein:
   at least one of R1 to R4 has a formula X—Y, wherein Y is a peptide sequence providing cell penetration, and X is absent or is a chemical linker;
   the or each of the remaining R1 to R4 groups are, independently, lipophilic, uncharged chemical groups; and
   Me is selected from Pt²⁺ or Pd²⁺,
      which probe is capable of measurement of molecular oxygen within live respiring cells by quenched-phosphorescence detection.

The term “phosphorescent analogs” should be understood to mean phosphorescent derivatives of the compound of Formula I in which the central phosphorescent moiety is replaced with an alternate porphyrin-based phosphorescent moiety, such as coproporphyrin III, coproporphyrin-1-ketone, and tetra(p-carboxyphenyl)porphine or closely related tetrapyrollic structures. Specific examples of phosphorescent analogs include:

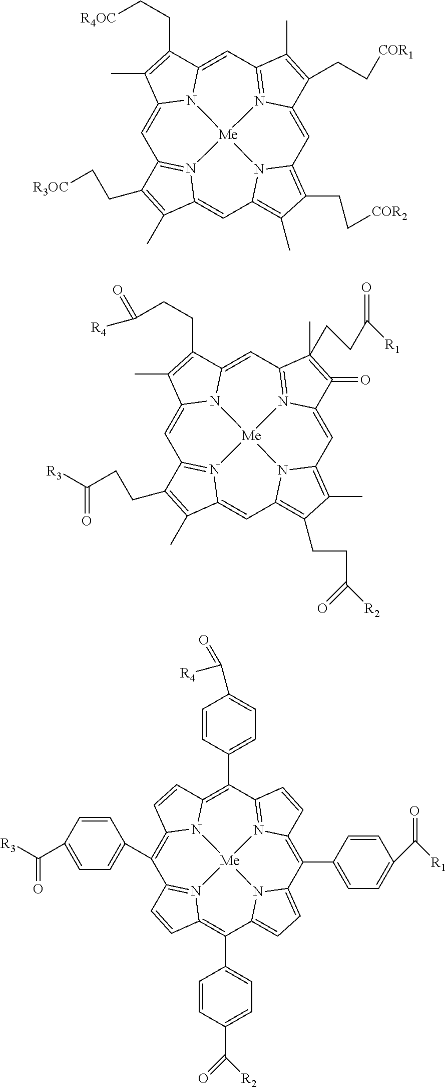

The compounds of the invention may be generally linear, asymmetrical molecules, in which one of R1 to R4 has the formula X—Y, and the remaining three of R1 to R4 are, independently, lipophilic, uncharged chemical groups.

Typically, R1 is X—Y, and R2 to R4 are, independently, uncharged chemical groups. Various examples of uncharged, chemical groups will be known to the person skilled in the art, for example alkoxy and ethylene glycol groups. In a preferred embodiment, the uncharged, chemical group is a lower alkoxy group, for example a C1-C4 alkoxy group. Typically, it is selected from a methoxy or ethoxy group.

The invention therefore relates to a compound of the invention, and having the general formula II:

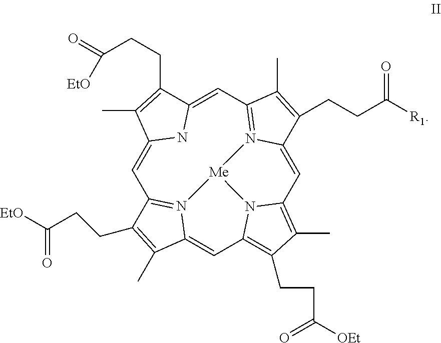

When the compound is a linear molecule, the peptide sequence providing cell penetration will generally be a cell penetrating peptide; that is to say, the peptide itself is capable of penetrating a cell. Thus, in one embodiment, Y is a cell penetrating peptide sequence selected from the group consisting of: CFRRRRRRRRRR (SEQUENCE ID NO: 1); F/GRRRRRRRRR (SEQUENCE ID NO: 2/7); GPRPLPFPRPG (SEQUENCE ID NO: 3); CFGRKKRRQRRR (SEQUENCE ID NO: 4); or functional variants thereof. Examples of alternative cell penetrating peptides will be known to the person skilled in the art, and from the literature, some examples of which are provided below.

In one embodiment of the invention, the chemical linker X is a common chemical linker. Examples of such common chemical linker structures will be well known to those skilled in the art, and include chemical linkers based on maleimide, pentafluorophenyl, N-succinimide, or isothiocyanatophenyl moieties. The chemical structures of some of these linkers are provided in FIG. 1 below. In an embodiment of the invention in which X—Y is maleimide-Y or PEG-maleimide-Y, the cell penetrating peptide Y typically includes a cysteine residue, and the linker is conjugated to Y via a thiol linkage to the cysteine residue. The cysteine residue may located at any position within the cell penetrating peptide, for example at either end or intermediate the ends.

Typically, the compound of the invention has a chemical structure selected from the group consisting of:

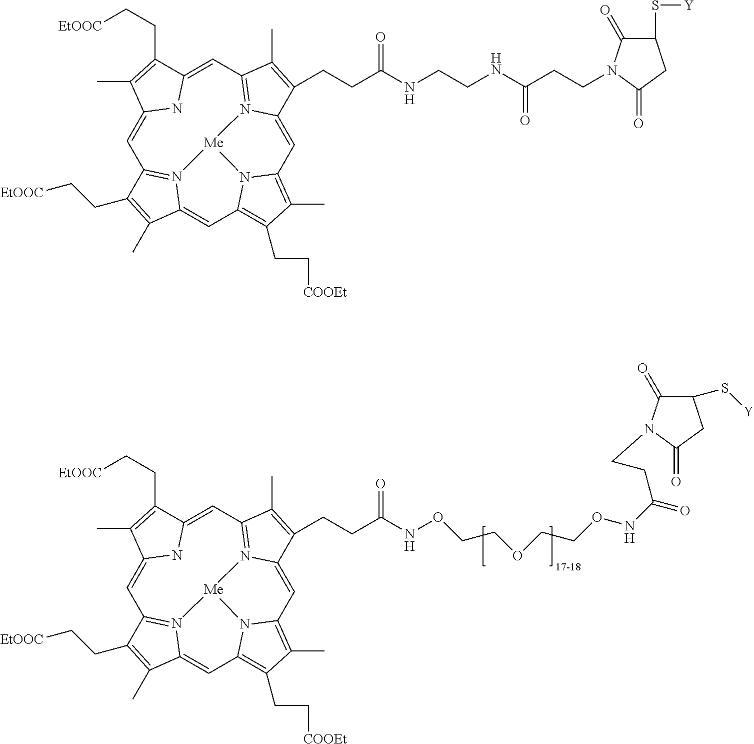

In the examples above and below, the linker is shown attached to a sulphur atom of a cysteine residue of the cell penetrating peptide (Y). The compounds above and below are triethyl ester derivatives, however it will be appreciated that the compound of the invention may also be a trimethyl ester derivatives.

Suitably, the compound of the invention has a chemical structure selected from the group consisting of:

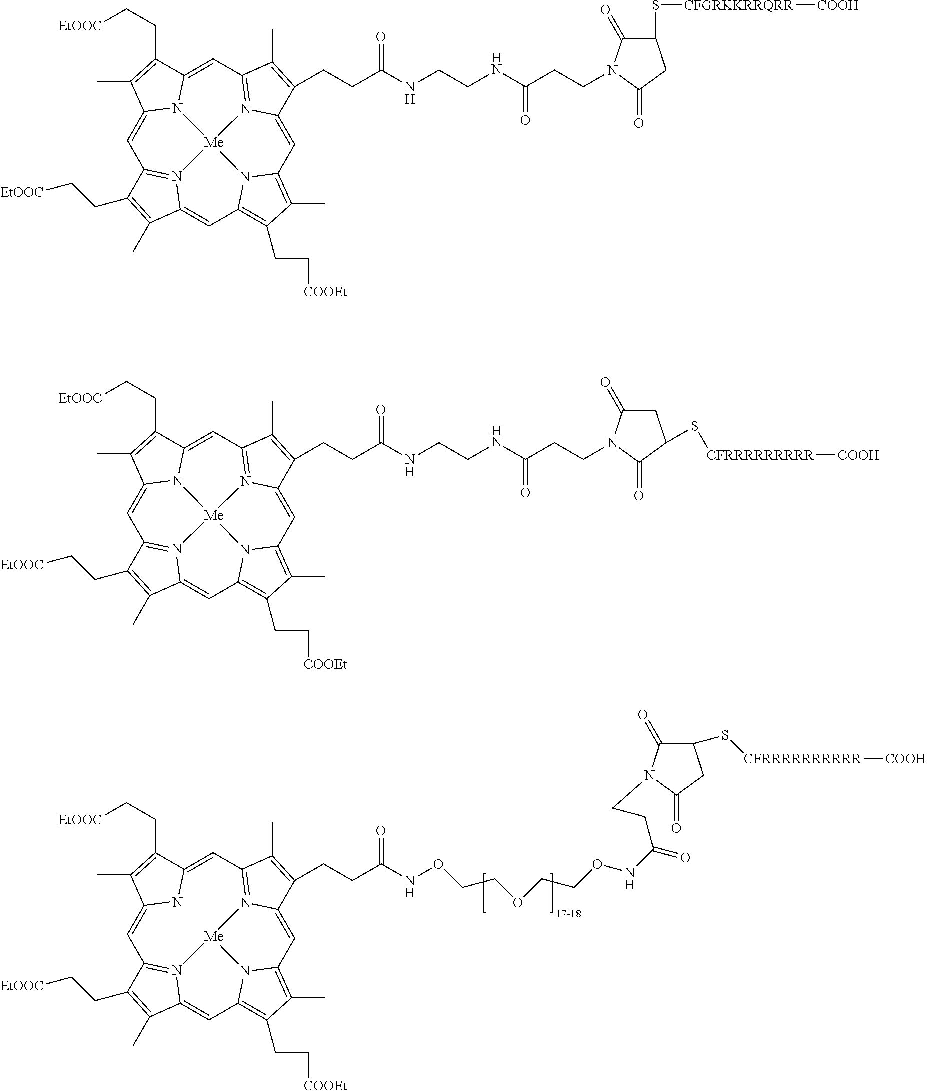

In another embodiment, the compound of the invention has a chemical structure selected from the group consisting of:

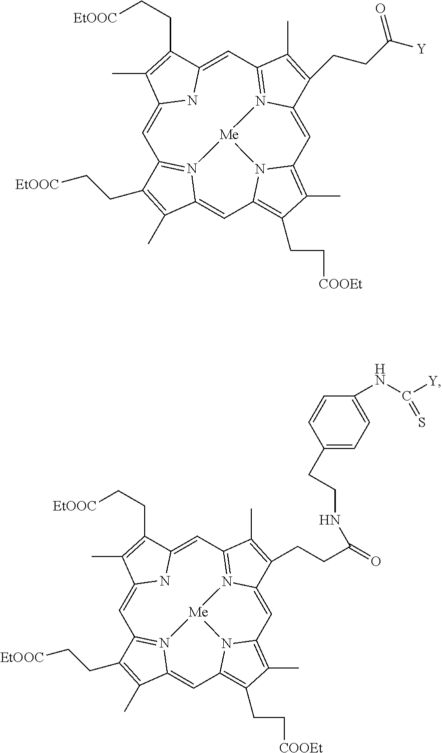

Typically, the compound has a chemical structure selected from the group consisting of:

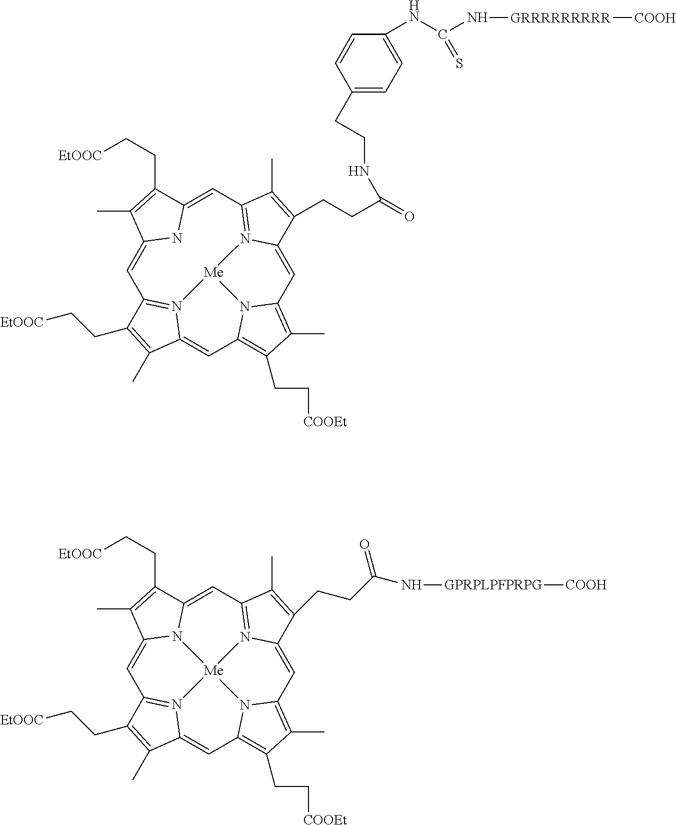

In another embodiment of the invention, the compound has a more symmetrical structure. In this embodiment, the porphyrin moiety is derivatised with four peptide sequences providing cell penetration (Y), typically four short, cationic, peptides (Y). Taken together, these peptides provide cell penetrating capability to the compound. Thus, typically each of R1 to R4 is Y, and in which Y is a cationic peptide containing less than five amino acid residues.

Suitably, the cationic peptide bears at least two arginine residues. Preferably, the cationic peptide comprises or consists essentially of a di-arginine moiety. Preferably, the cationic peptide is di-arginine amidated at C-terminus and linked via its N-terminus.

Typically, the compound of the invention has a chemical structure selected from the group consisting of:

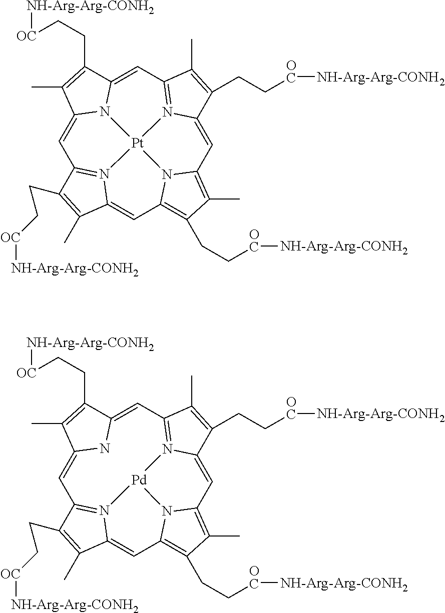

In one embodiment, the cell-penetrating peptide sequence Y is capable of targeting the probe to a specific location within the cell, for example, mitochondria, late endosomes, lysosomes, endoplasmic reticulum. Suitably, Y is capable of targeted the compound to mitochondria, wherein Y has the sequence: MGRTVVVLGGGISGLAAGCGRRRRRRRRR (SEQUENCE ID NO: 5) Ideally, the peptide sequence is linked via the internal cysteine residue (MGRTVVVLGGGISGLAAGCGRRRRRRRRR).

The invention also relates to the use of a compound of the invention as a probe for measurement of oxygen within live respiring cells by quenched-phosphorescence detection.

The invention also relates to a method of assessing the oxygen status of live cells, which method includes the steps of:
a. providing a sample of cells;
b. exposing the cells to a phosphorescent compound of the invention to load the cells;
c. optionally, removing excess phosphorescent compound from extracellular medium;
d. measuring a phosphorescent signal of one or more cells loaded with probe; and
e. correlating the phosphorescent signal with oxygen status within the cell(s).

Suitably, the method of the invention is a method of assessing absolute or relative oxygen level/concentration of one or more live cells.

Suitably, the phosphorescent signal from the loaded cells is measured by a technique selected from the group consisting of: steady-state fluorometry; time-resolved fluorometry; phase fluorometry; phosphorescence lifetime measurements; and fluorescence imaging.

Preferably, the phosphorescent signal is measured in a form of intensity, lifetime or phase shift.

The phosphorescent signal may measured for the whole population of cells, an individual cell, or a particular sub-cellular location.

Suitably, the oxygen level/concentration is quantified using pre-determined calibration of the probe.

In one embodiment, the cells are loaded by simple incubation of the cells with phosphorescent compound.

The invention also relates to a method of synthesis of a compound according to the invention, which method employs a heterofunctional derivative of Pt- or Pd-coproporphyrin I.

Typically, the method of synthesis employs a derivative of Pt- or Pd-coproporphyrin I containing at least one reactive chemical group that facilitates conjugation of peptide sequence Y.

Suitably, the method includes a step of generating the cell penetrating peptide sequence by solid-phase peptide synthesis or by recombinant protein technology.

The invention also relates to a phosphorescent compound obtainable by a method of synthesis of the invention.

### BRIEF DESCRIPTION OF FIGURES

FIG. 1: Probe chemical structures. The major substituent positions designated as R1-R4. Me is the ion of Pd (II) or Pt (II) coordinated by the porphyrin ring. In monofunctionalyzed MeCP-TE derivatives R2=R3=R4=OC₂H₅; in MeCP derivatives R2=R3=R4=OH; Structures of common linkers used for conjugation are also shown: NCS, maleimide PFP.

FIG. 2: Examples of synthesized cell penetrating conjugates

FIG. 3: Comparison of loading properties of different O₂ probes. Average fluorescent signals from PC12 cells loaded with probes for 16-24 hrs. Abbreviations: PEPP2—PTCPTE-PEG-CFR9; PEPP3—PtCP-R2. For comparison, signals of the PtCP-BSA probe loaded with Endo-Porter agent are also shown.

FIG. 4: Images showing sub-cellular localization of the O₂ probes in SHSY-5Y human neuroblastoma cells obtained by live cell microscopy. Brightfield image; fluorescence images of MitoTracker Green probe (MTG); fluorescence images of different oxygen probes measured under 390 nm excitation and 650 nm emission. Probe abbreviations: PEPP1A—PTCPTE-CFR9 probe; PEPP2—PTCPTE-PEG-CFR9 probe; PEPP3—PtCP-R2 probe; PEPP4—PTCPTE-PEG-MTS-CGR9 probe; PEPP5—PTCPTE-MTS-CGR9 probe. Cell were loaded with O₂ probes for 16 hrs in regular medium, washed then loaded with MTG for 15 min and imaged.

FIG. 5: Phosphorescence lifetimes of the intracellular probe. PTCPTE-CFR9 at different pO2 in the hypoxia chamber. The difference between the respiring and non-respiring cells (treated with Antimycin A) illustrates the presence of localized O₂ gradients in respiring cells.

The results obtained with the non-respiring cells can be used as calibration function to convert measured lifetime values into O₂ concentration.

FIG. 6: Monitoring of respiratory responses to cell stimulation. PC12 cells were plated in standard 96-well plates at 50,000 cells/well, then loaded with 10 μM PtCPTE-CFR9 probe for 16 hrs, washed and measured on a time-resolved fluorescence plate reader Victor2. After baseline stabilization, the following compounds were added: DMSO (blank), 1 μM FCCP, 10 μM antimycin A, 0.5 μM valinomycin or 100 mM KCl. The observed responses of the probe reflect changes in cell respiration and local O₂ concentration in loaded cells.

### DETAILED DESCRIPTION OF THE INVENTION

Considering the problems with existing probes highlighted above and general practical requirements, the development of new intracellular O₂ probes of the invention was focused around supramolecular structures comprising covalent conjugates of the phosphorescent Pt- and Pd-porphyrins with cell-penetrating peptides and similar hybrid structures. To determine optimal structures of such probes, a range of different phosphorescent dyes, peptide sequences, conjugation chemistries and site-specific modifications were tested and investigated in detail.

Initially, probe design was focused on Pt- and Pd-coproporphyrin (MeCP) dyes, which were used as the phosphorescent O₂-sensitive moiety of the probe. MeCP also contains four propionic acid residues as side substituents which make the structure hydrophilic. These carboxylic groups deprotonate at physiological pH producing a significant negative charge, MeCP dyes are not cell-permeable and can not be used as intracellular probes. However, MeCP dyes are well suited for chemical modifications, derivatization and conjugation with different chemical and biological structures.

For example, monofunctionalised labelling reagents on the basis of MeCP have been developed, particularly phenylisothiocyanato and maleimido derivatives of PtCP and PdCP (U.S. Pat. No. 6,582,930, 2002). They were used for the synthesis of phosphorescently labelled oligonucleotides, proteins and polypeptides (Papkovsky D B, O'Riordan T C. J Fluoresc. 2005; 15(4):569-84) which were then applied to different bioassays. PtCP-BSA and PtCP-PEG conjugates were used as probes for sensing O₂ (Hynes, J., et al. J Biomol Screen., 2003, 8(3): 264-72; O'Donovan C., et al. J. Material Chem. 2005, 15(27-28): 2946-2951), mainly extracellularly but also intracellularly in combination with cell loading reagents which provided their transport into the cell (O'Riordan T C, et al.—Am J Physiol Regul Integr Comp Physiol. 2007; 292(4):R1613-20; Papkovsky D B, et al—EP2043694 (A2), 2009). But again, such conjugates possess no ability to penetrate cell membranes and accumulate in predetermined locations within the cells.

The second important component in the design of intracellular O₂ probes of the invention was the available information about the structures and properties of cell-penetrating molecules, particularly of peptide nature, the mechanisms of their transport across plasma membrane of mammalian cells and other intracellular compartments, and the factors determining transport of various substances into the cells. Cell-penetrating peptides have been used to deliver into the cell various chemical and biological specie ranging from small molecules to large proteins (Foerg, C. et al.—J Pharm Sci 97:144-162; 2008). Thus, TAT peptide and its oligoarginine analogs (8-10 residues) have demonstrated high efficiency in cell penetration, they were efficient in loading the cells with various molecules ranging from small fluorophores to high molecular weight proteins such as b-galactosidase. Conjugates of porphyrin dyes with TAT peptides developed for photodynamic tumour therapy are also known (Sibrian-Vazquez M. et al.—Bioconjug Chem. 2005, 16:852-863; and Bioconjug Chem 2006, 17:928-934; Choi Y et al.—Chem Med Chem 2006, 1:458-463), however they can not be used as O₂ probes and their main function is to kill the cells. The mechanisms of cellular uptake for TAT conjugates are thought to be different, ranging from direct translocation to macropinocytosis. The ability to penetrate cell membrane depends on the total positive charge provided by guanindine groups rather than on the type of linkage to the peptide (N or C terminus). Among other structures, penetratin and bactenecin families having good cell penetrating efficiency, can also be regarded as good candidates for the development of intracellular O₂ probes.

Based on these considerations, the following peptides were selected for the synthesis of cell-penetrating O₂ probes: TAT (48-60), R₉ and BN (bactonecin 715-724). To increase peptide absorbance in the UV, one phenylalanine residue was added to TAT and R₉ peptides. For selective labeling with maleimide derivatives, cysteine residues were incorporated N-terminally to these peptides. Relatively short length of these structures (8-10 amino acids) allows production of conjugates in high yields and purity by peptide synthesis technologies. Resulting peptide sequences and their abbreviations are given in Table 1.

TABLE 1
List of peptide sequences tested

| | | Sequence |
|---|---|---|
| No. | Name of peptide | (one letter code) |
| 1 | CFR9 | CFRRRRRRRRRR |
| | (nonaarginine) | (SEQ ID NO: 1) |
| 2 | FR9 | FRRRRRRRRR |
| | (nonaarginine) | (SEQ ID NO: 2) |
| 3 | BN (bactenecin) | GPRPLPFPRPG |
| | | (SEQ ID NO: 3) |
| 4 | CTAT (TAT) | CFGRKKRRQRRR |
| | | (SEQ ID NO: 4) |
| 5 | R2 | RR-NH₂ (C-terminal amidation) |
| | | (SEQ ID NO: 6) |
| 6 | MTS-CGR9 | MGRTVVVLGGGISGLAAGCGRRRRRRRRR |
| | | (SEQ ID NO: 5) |

Following the design of the cell-penetrating peptides, their phosphorescent labelling with MeCP dyes was undertaken aiming at producing O₂ probes for intracellular use. Initially, PtCP-NCS-derivative was used to label BN and FR9 peptides via N-terminal amino groups. This method was achieved by simple mixing and incubation of the two components followed by conjugate separation by HPLC. Pure conjugates were synthesised in sub-micromolar quantities and characterized by spectroscopy.

When these conjugates were examined with cells, their cell-penetrating ability was low. We attributed this to the anionic nature of the porphyrin label (PtCP-NCS) and its negative charge (−3) preventing the conjugate from going into the cell. The conjugation between the negatively charged PtCP-NCS and positively charged R9 peptides was also associated with precipitation, which reduced the yield of conjugation.

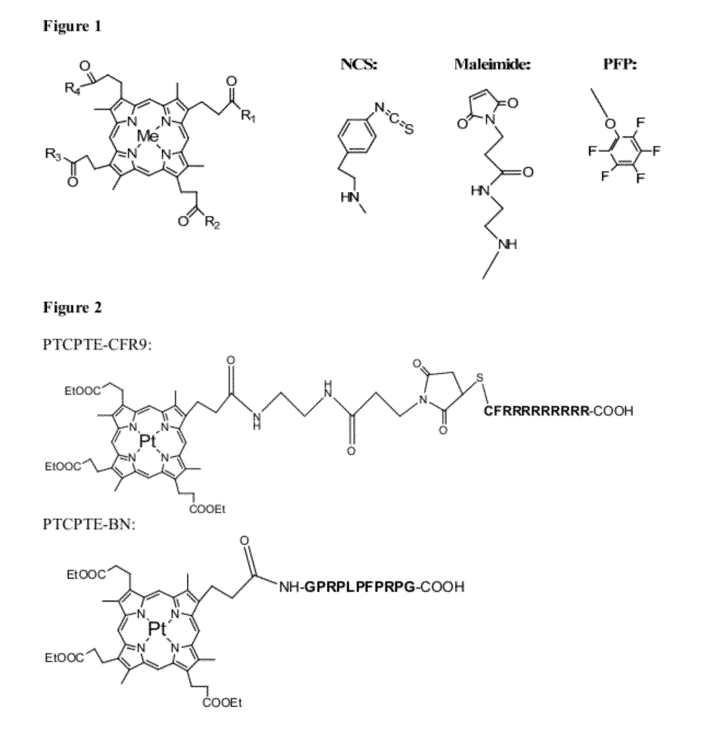

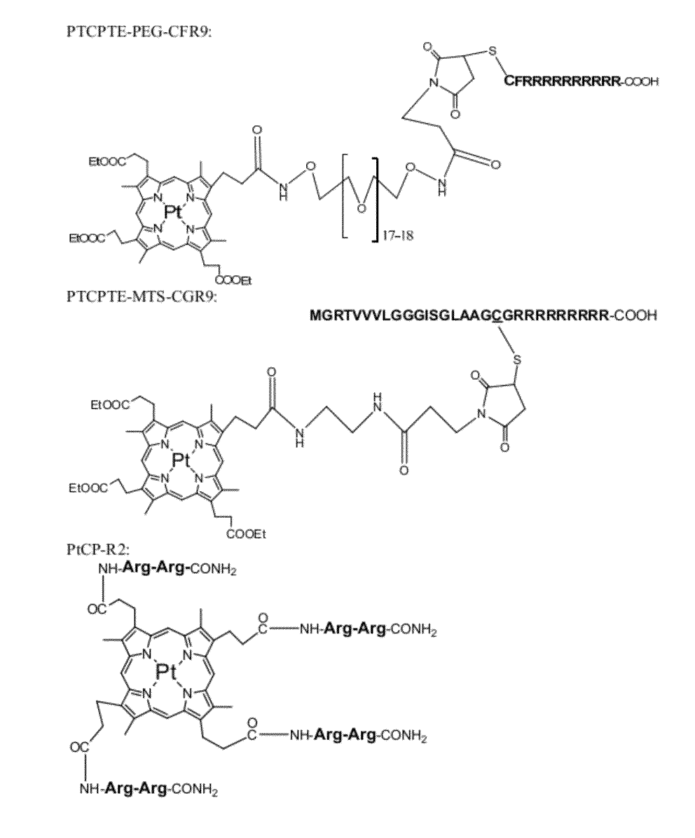

To overcome these problems and increase cell-penetrating ability of the conjugates, the structure of MeCP label (FIG. 1-2) was modified. New triethyl ester derivatives of monofunctionalised PtCP, namely maleimido (PTCPTE-MI), pentafluorophenyl (PTCPTE-PFP) and isothiocyanato (PTCPTE-NCS) were prepared and used for labelling of cell-penetrating peptides.

When CTAT and CFR9 peptides were conjugated with the neutral PTCPTE-MI dye, cell loading properties of resulting probes were seen to improve significantly compared to those of the PtCP conjugates. Both PTCPTE-CTAT (overall charge +5) and PTCPTE-CFR9 (charge +8) conjugates showed fast and high loading of dPC12 cells, with fluorescent signals ranging 60,000-200,000 cps (counts per second) after 2-24 h of loading. Despite of the equal charge, the PTCPTE-CFR9 conjugate demonstrated a higher intracellular uptake than PTCPTE-TAT.

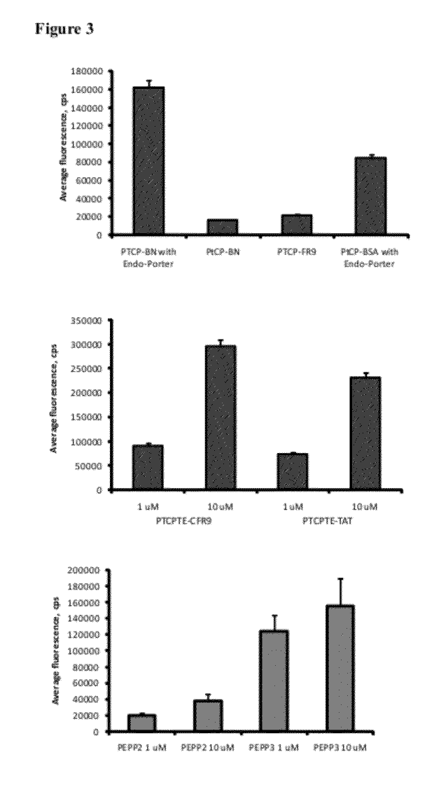

These results illustrated that spatial distribution of positive and negative charges plays important role in cell penetrating ability of the probe, rather than the overall positive charge. Also the conjugates with negative charges localized at the fluorophore moiety were less efficient in cellular uptake. After 24 h of loading, average fluorescence signals of about 200,000 counts for both PTCPTE-CTAT and PTCPTE-CFR9 probes, were significantly higher than for PtCP-BSA probe loaded by transfection (60,000 cps) (FIG. 3). Higher phosphorescent signals provided by the new cell-penetrating probes resulted in improved performance of O₂ sensing experiments. These probes allowed more reliable and accurate determination of the phosphorescence lifetime and cellular O₂ concentration, more stable baseline produced by the resting cells and better sensitivity and resolution in detecting small changes in cellular respiration. Cell loading time can be shortened to 1-2 hours.

Furthermore, for all the probes with high cell loading efficiency, a very low cytotoxic effect was observed with different cells. After 24 h loading with PtCP-FR9, PTCPTE-CTAT and PTCPTE-CFR9 probes, cell viability was in the range 93-95%, which is better than for conventional probes loaded by transfection (O'Riordan T C, et al.—Am J Physiol Regul Integr Comp Physiol. 2007; 292(4):R1613-20). Negligible effect on cell viability allows the use of these conjugates in cell physiology studies, including long-term experiments.

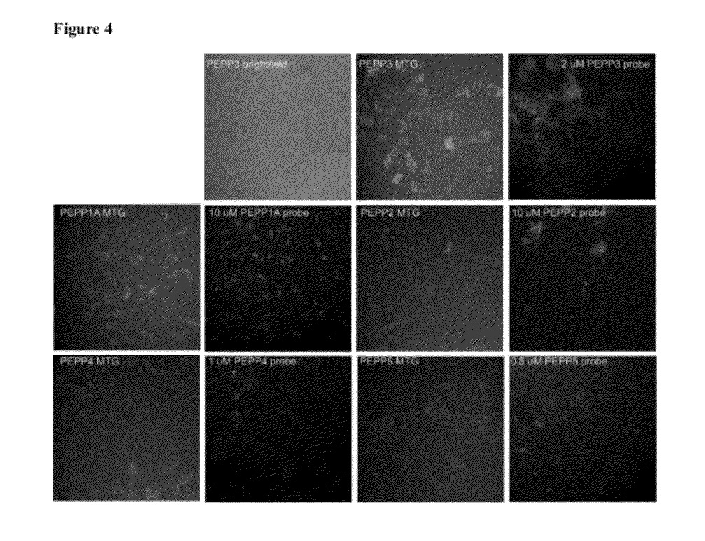

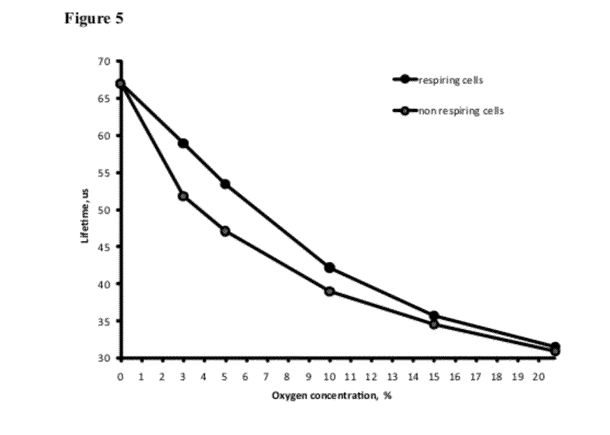

The new cell-permeable O₂ probes were then calibrated at different concentrations of external O₂ (FIG. 5). In one embodiment, PC12 cells were loaded with PTCPTE-CFR9 probe and exposed to different pO₂ levels ranging from normoxia (20.8% O₂) to anoxia. Phosphorescence lifetime of the probe were measured in both respiring and non-respiring (treated with antimycin A) cells (FIG. 4). For the non-respiring cells, average lifetime under normoxia was 33±2 us, and increased to 65 us in deoxygenated conditions. This was similar to the characteristics of the conventional (cell-impermeable) O₂ probe PtCP-BSA (O'Riordan T C et al.—Anal Chem 2007, 79:9414-9419), and is considered as optimal for given application.

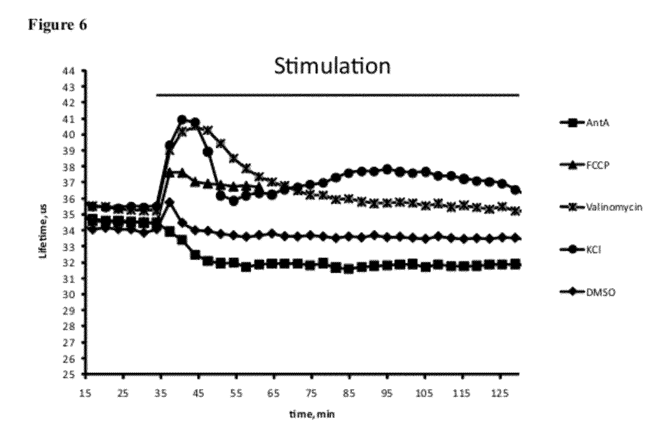

Following the initial physical chemical evaluation of the new cell-penetrating O₂ probes, they were applied to the analysis of cellular responses (FIG. 6). The uncoupler of oxidative phosphorylation protonophore FCCP increases O₂ consumption of PC12 cells. The respiratory response to stimulation with 1 uM FCCP was observed for PtCP-FR9, PTCPTE-CFR9 and CTAT probes, which had a magnitude of about 2 microseconds in lifetime units. Notably, even after 2-6 hrs of loading with 10 uM of PTCPTE-CFR9, stable lifetime readings and easily detectable responses to stimulation by FCCP were seen. Other known drugs: antimycin A (inhibitor of the electron transport chain at complex III), potassium ionophore valinomycin (uncoupler of oxidative phosphorylation) and potassium chloride (membrane depolarizing agent) also generated easily measurable responses which were in agreement with their mode of action on cell respiration. Using these probes with certain cell types, loading time and preparation of cells for the assay can be reduced to 1-2 hrs.

The uptake of the new O₂ probes by different cell lines was investigated. All the PTCPTE-based probes used at working concentration of 10 uM were found to load efficiently HepG2, HCT116, Hela and SHSY5Y cells, average TR-F signals exceeded 60,000 cps. Probe loading worked well with both adherent and suspension cells, and in different media with high and low protein content. In all these cases, loaded cells gave marked responses to stimulation with model drugs. This shows that, unlike with conventional probes, cellular uptake of PTCPTE conjugated to TAT derivatives is normally high and not dependent on the cell and medium used. The PTCPTE-based peptide probes were also efficient in loading live mammalian tissues (tissue slices cultured in growth medium). In contrast, PtCP-BN and PtCP-FR9 conjugates harbouring the negatively charged porphyrin, showed low uptake by PC12 cells and ther did not load well HCT116 and other cells tested.

The intracellular localization of the probes was then investigated by live cell fluorescence microscopy. FIG. 4 shows that in SHSY5Y cells the PTCPTE-CFR9 probe is localized mainly in the cytoplasmic dot-like structures. Some aggregates were seen outside the cells. In HeLa, PC12, HepG2 and HCT116 cells localisation pattern was similar, giving slightly higher probe distribution in the perinuclear region. The probe did not co-localize with the mitochondria and was located close to the lysosomes, although no full overlap with LysoTracker Green was seen. Without being bound by theory, it can be concluded, that this probe localizes in the compartments of secretory pathway such as endosomes or trans-Golgi network. No significant co-localization with LysoTracker suggests that the O₂ probe was not subjected to lysosomal degradation after cellular uptake. A similar subcellular localization was observed for the PTCPTE-TAT probe. Such localization reflects rather uniform cellular uptake of the probe through endocytosis, rather than via direct translocation.

The above results demonstrate that the conjugates of cell-penetrating peptides with uncharged PtCP moiety, work effectively as cell-penetrating O₂ probes. They spontaneously accumulate in cells where they display bright phosphorescence easily detectable by fluorescence imaging or by measurement on a time-resolved fluorescence plate reader. Moreover, these probes showed significantly higher loading efficiency and faster loading rates than conventional probe loaded with the aid of transfection reagents (PtCP-BSA—O'Riordan T C et al.—Anal Chem 2007, 79:9414-9419) or by other facilitated means. They stayed within the cells for long periods of time without any significant loss of intracellular location over time. Their sensing properties and calibration were quite optimal for sensing O₂ in respiring cells under physiological conditions (both normoxia and hypoxia). They can be successfully used to study intracellular O₂ levels in live respiring cells and for monitoring of dynamic changes in cell respiration upon stimulation, by simple means.

In a similar way, the conjugates of BN and some other cell-penetrating peptides with the uncharged PTCPTE labels were prepared, using labelling via N-terminal amino group (with PFP-derivative) and via the SH-groups of cystein residues (with MI-derivatives). They all showed high cell loading efficiency, similar to the CFR9 conjugate and usability as probes for cellular O₂ with self-loading capabilities. In addition, we prepared similar conjugates with PdCPTE derivatives. As expected, the resulting conjugates showed a similar cell-loading behaviour as the conjugates of PTCPTE, but different spectral properties and higher sensitivity to O₂. These probes are more suitable for work at lower O₂ concentrations (deeper hypoxia). Structures of some preferred probes of the invention which have proven high performance in O₂ sensing experiments with cells are given in FIG. 2.

Furthermore, having succeeded with the development of cell-permeable O₂ probes, we attempted the development of even more advanced probes, particularly the probes targeted to specific sub-cellular compartments. This was achieved by modifying the sequence of cell-penetrating peptide with an additional sequence of a leading peptide which is known to provide delivery and specific localization of within the cell for particular proteins produced endogenously by the cell. One such peptide, which contained the mitochondria targeting sequence from human protoporphyrinogen oxidase (PPO) combined with the cell-penetrating sequence, and O₂ probe on its basis are shown in Table 1 (MTS-CGR9). This sequence was successfully labelled with PTCPTE-MI derivative. The resulting probe was seen to retain high cell loading efficiency. Notably, its cell-penetrating ability was not compromised by the extended peptide sequence, and its accumulation in dPC12 and other cell lines was similar to that of the PTCPTE-CFR9 probe. At the same time, this probe showed preferential co-localisation with mitochondria, as confirmed by fluorescent imaging with staining the cells with the O₂ probe, MitoTracker Green or protein based Ca²⁺ sensor mitoCase12. For the specialists in the area, it is clear that a similar approach can be used to direct the cell-permeable O₂ probes to the other cellular locations (i.e. nuclei, whole cytoplasm etc), by incorporating (N- or C-terminally) the appropriate targeting sequences in probe structure.

One limitation of cell-penetrating O₂ probes based on MeCPTE is their relatively high hydrophobicity associated with the label. The limited solubility of MeCPE-NCS and MeCPE-MI labels complicates probe synthesis and results in elevated non-specific signals in the experiments with cells (probe binding to surfaces). This does not prevent the use of the probes (non-specific binding can be reduced by loading cells in suspension with subsequent washing and seeding loaded cells on assay substrate), but may cause undesirable complications in interpreting the results. The present invention addresses these issues by providing several modifications of the probes, using more hydrophilic but still neutral and small size phosphorescent MeCP labels. Thus, labelling reagent was designed containing a hydrophilic polyethylene glycol (PEG-850) spacer PTCPTE-PEG-MI, which was found to facilitate probe synthesis. The resulting probe showed a similar cell-loading and O₂ sensing behaviour as the probes based on PTCPTE-MI label, but higher hydrophilicity.

Furthermore, in the course of the extensive experiments with phosphorescent supramolecular structures, their physical-chemical and cell-penetrating properties, and with loading mammalian cells with such structures, yet another type of intracellular O₂-sensitive probe was generated. The previously described probes of the invention comprised derivatives of MeCP modified with one relatively long cell-penetrating peptide (10 or more amino acid residues) bearing multiple positive charges. In contrast to these mono-substituted, linear, polycationic probe structures, a number of alternative probes were generated. These probes are based on the same phosphorescent metalloporphyrin moiety which is poly-substituted with cationic groups to form the symmetric, non-linear structures. A number of such conjugate structures were synthesised and tested for their cell-penetrating ability and suitability as intracellular O₂ probes.

The first phosphorescent structure of this type was based on the PtCP dye in which all four propionic acid residues were modified with the diarginine peptide. On their own, both PtCP dye and diarginine (dicationic peptide) do not show cell-penetrating ability. Previously it was shown that cell-penetrating ability of oligoarginine peptide depends not so much on the sequence of amino acids, but on the total number of arginine residues and positive charges (Futaki S et al.—Biochemistry 2002, 41:7925-7930). Tetra-substituted diarginine derivatives of PtCP with a total number of arginines of 8 were then tested. It was found that such structure (shown in FIG. 1) does show good cell-penetrating ability. Moreover, this structure does not display any toxicity (previously seen for the other cationic porphyrins), and it behaved very similar to the first group of O₂ probes based on longer cell-penetrating peptides like PTCPTE-CFR9 (see above). Moreover, compared to the first type of the probe of the invention, this probe is more hydrophilic, can work at lower concentrations and has more cytoplasmic localization within the cell.

Using similar general design, other modifications of O₂ probes were synthesised, such a PdCP-based diarginine probes which are more suited for use at hypoxic conditions. By varying peptide residues, their charge and hydropathy, an extended panel of such symmetric, poly-substituted MeCP based probes was developed. The invention also demonstrates that using the same strategy, such probes can also be targeted to particular sub-cellular locations.

When a different cationic dye—Pt-tetrakis(pyridinium)porphine tetrachloride was tested, it was seen to go into the cells, but it accumulated in the nuclei and showed high cyto- and phototoxicity on the cells which quickly lost their viability. Such behaviour is likely to be linked to the dye interaction with nucleic acids (intercalation described for such porphyrin structures) causing interference with cellular function. This structure is therefore unusable as probe for cellular O₂. We also synthesised and tested cationic tri-amino and tetra-amino derivatives of PtCP having total molecular charges +2 and +4, however these dyes showed very low intrinsic cell-loading ability with several different cell lines tested.

The above examples illustrate that the development of cell-penetrating probes based on phosphorescent dyes poly-substituted with cationic groups was also achieved. This type of probe can also be used effectively as intracellular O₂ probes and can also be targeted to specific sub-cellular locations. Compared to the first probe type, the probes are easier and cheaper to produce.

Furthermore, probe sensitivity to O₂ and photophysical properties can be adjusted by changing the structure of the central phosphorescent moiety. Thus, by replacing PtCP with PdCP moiety, it was possible to increase probe sensitivity to O₂ several-fold. By replacing PtCP with PtCP-ketone (Papkovsky D B, Ponomarev G V U.S. Pat. No. 5,718,842, 1998), it was possible to increase probe photostability and shift spectral characteristics towards longer wavelengths.

Overall, the invention provides a family of advanced O₂ probes for the measurement and continuous monitoring of O₂ within live mammalian cells. These probes combine a number of important features which make them superior to most of the existing O₂ probes developed for similar applications. These features are: facile and efficient delivery into the cell and, if required, targeting to a specific location within the cell; minimal invasiveness, low cyto- and phototoxicity and interference with cellular function; minimal leakage from the cell and subcellular compartments; convenient photophysical properties, optimal sensitivity and selectivity to O₂; well-defined chemical composition, simple procedure of probe synthesis, good storage and operational stability; excellent compatibility with different types of cells, tissue and measurement conditions (media additives, etc); flexibility which allows fine-tuning probe properties and composition and development of new modifications of these probes by relatively simple chemical and biological means.

The invention provides a basis for rational design of two new types of intracellular O₂ probes based on the phosphorescent metallopoprhyrin moiety, namely, the linear monosubstituted conjugates with relatively long polypeptides, as well as the symmetric, poly-substituted conjugates with short cationic peripheral groups. The ease of manufacturing, convenience of use, flexibility and robustness of the probes of the invention and bioassays on their basis are other important advantages. The probes can be directed to specific locations within the cells, particularly the mitochondria where cellular O₂ is mainly consumed. These new chemistries open opportunities for probing of localised O₂ gradients in respiring cells and tissues, and for the analysis of cell respiration, disease states and metabolic perturbations, (patho)physiological conditions such as hypoxia, cellular responses to stimulation and drug treatment. Compared to the existing (macro)molecular and carrier-based O₂ probes which require facilitated transport into the cell, the new peptide probes do not require any additional reagents, treatment or transfection steps, while they provide fast and efficient passive loading of different cells.

The invention also provides new methodologies for the measurement of (sub)cellular O₂ which are based on the use of these probes, as well as the use of these methodologies in a number of core biological applications and measurement tasks in which quantification, continuous monitoring and imaging of cellular O₂ represent the main goal.

The invention is illustrated with the following non-limiting examples.

### Example 1

### Synthesis of the Probes Based on PtCP Derivatives Conjugated to Cell-Penetrating Peptides

The amino-coupling conjugations (PtCP-NCS with FR9 and BN, PTCPTE-PFP with BN, PTCPTE-NCS with FR9, PTCP-tetraPFP with R2 and others) were performed in DMSO containing triethylamine (2 ul per 100 nmole reaction), using 1:1 peptide/porphyrin molar ratio (4:1 for R2), and 24 h incubation at room temperature. The thiol-coupling conjugations (PtCPTE-MI or PTCPTE-PEG-MI with Cys-containing peptides CTAT, CFR9, MTSCGR9) were performed in DMSO, using molar ratio peptide/porphyrin 2-4:1 and incubation at room temperature for 24-72 h. The reactions were monitored by HPLC on an analytical C18 reversed phase column using a gradient of acetonitrile in 0.1% aqueous trifluoracetic acid (TFA). Conjugates were purified on a preparative column using the same conditions. Purified conjugates were quantified by absorbance measurements at the Soret band (maximum at about 380 nm), then vacuum-dried and stored at −18° C. For experiments they were resuspended in DMSO or loading medium (e.g. RPMI1640 medium supplemented with 1% of horse serum). Yields for amino-coupling and 66-100% for maleimide-coupling conjugations were ˜100% with respect to the dye. The structures of the synthesized probes are given on FIGS. 1-2.

### Example 2

### Assessment of Cell Loading with Different Probes by Time-Resolved Fluorometry

PC12 cells were seeded in standard 96-well plates pre-coated with collagen IV at 5×10⁴ cells/well, and differentiated for 3-5 days in RPMI supplemented with 1% horse serum, P/S, and 100 ng/ml nerve growth factor. HepG2 cells were cultured in DMEM supplemented with 10% FBS, 2 mM L-glutamine and P/S, CHO—in Ex-Cell CHO DHFR⁻ medium supplemented with 4 mM L-glutamine and 1 μM methotrexate, HCT116—in McCoy medium supplemented with 10% FBS, 2 mM L-glutamine and P/S. The samples with cells were then loaded by incubating them with 1-10 μM of the O₂ probe (prepared as described in Example 1) in regular medium at 37° C., 5% CO₂, for the required period of time. After incubation the cells were washed 3 times with medium and 0.1 ml of fresh medium were added to each well. For comparison, some samples with cells were loaded with 1.2 μM PtCP-BSA probe (Luxcel Biosciences) in the presence of 6 μM Endo-Porter, as previously described (Zhdanov A V et al—J Biol Chem 283:5650-5661; 2008). The plate with loaded cells and control samples was then measured on a time-resolved fluorescence (TR-F) plate reader Victor 2 (PerkinElmer) set at 37° C., using 340 nm excitation and 642 nm emission filters. Each well was measured several times by taking TR-F intensity reading at delay time of 30 μs and gate time 100 μs. Measured TR-F signals were averaged and used to assess the efficiency of loading. Representative loading patterns are shown in FIG. 3.

### Example 3

### Analysis of Sub-Cellular Localisation of the Probes by Live Cell Fluorescent Imaging

PC12, Hela, HepG2, HCT116 and SH SY5Y cells were seeded onto 35 mm glass bottom imaging dishes at 25,000-30,000 cells/dish, typically 1 day before the experiment (3 days for PC12). Cells were loaded with 0.5-10 uM of peptide conjugates for 16-24 hrs, then washed three times with medium, counter-stained for 30 min with 100 nM of LysoTracker Green or 12.5 nM of MitoTracker Green and washed again. Microscopy analysis was conducted on a fluorescent microscope Axiovert 200 (Carl Zeiss) equipped with a custom-made LED module (LaVision) for excitation. An UV (390 nm) LED and “PtCP” (ex. 390/40 nm, em. 655/40 nm) filter cube were used for imaging of the PtCP-based probes (200 ms pulse length), whereas green (488 nm) LED and standard FITC filter cube were used for MitoTracker Green and LysoTracker Green dyes.

FIG. 4 shows that in SHSY-5Y cells PTCPTE-CFR9 (PEPP1A) and PTCPTE-PEG-CFR9 (PEPP2) probes are localized mainly in the cytoplasmic dot-like structures. The PtCP-R2 (PEPP3) probe is located diffusely in cytoplasm with some nucleolar accumulation, whereas mitochondria targeting peptide-containing probes PEPP4 and PEPP5 are located similar to the mitochondrial stain. Some aggregates were also seen outside the cells. In Hela, HepG2, dPC12 and HCT116 cells the patterns of location of the probes were similar to SHSY-5Y cells.

### Example 4

### Monitoring of Local O₂ Gradients in Mammalian Cells and Respiratory Responses to Cell Stimulation on a Time-Resolved Fluorescence Plate Reader

PC12 cells (seeded at 50,000 cells/well in standard 96 well plates and differentiated under standard conditions) were loaded with the cell-penetrating O₂ probes in standard medium, using 1-10 uM probe concentrations, volume 100 ul/well and incubation time ranging from 20 min to 24 hrs. After probe loading wells were carefully washed three times with DME medium without phenol red and containing NGF. The same protocol was used for loading of adherent HCT116, HepG2 and SHSY5Y cells, using corresponding media.

For the loading in suspension, non-differentiated PC12 cells were collected, passed through a syringe needle (size 22 gauge) ten times, counted and incubated with 10 uM of probe in RPMI1640 medium supplemented with 10% HS and 5% FBS for three hours, with mixing by pipetting every 30 min. Cells were centrifuged, washed three times with RPMI containing 1% HS and (at final wash) NGF, and seeded to wells of 96-well plate at 75,000 or 150,000 cells per well. Cells were allowed to attach for three hours and then processed to measurements. For comparison, PC12 cells were loaded with PtCP-BSA probe as described earlier (Zhdanov A V et al—J Biol Chem 283:5650-5661; 2008.), using 1.2 uM probe concentration, 6 ul/ml of Endo-Porter transfection reagent, and loading time 24-28 h at 37° C.

Measurement of the cells loaded with probes was conducted on a TR-F plate reader Victor 2 (PerkinElmer, USA) at 37° C. as described in (O'Riordan T C et al—Anal Chem 79:9414-9419; 2007), using 340 nm excitation and 642 nm emission filters. Each well was measured repetitively every 1.8 minutes by taking two TR-F intensity readings at delay times of 30 μs (T1) and 70 μs (T2), using gate time 100 μs. Fluorescence intensity values at T1 (F1) and T2 (F2) were used for calculation of lifetime values using the following equation:

Lifetime [us]=(T2−T1)/ln(F1/F2),

which were used to plot time profiles.

For cell stimulation, the plate was monitored for 10-20 min to reach O₂ and temperature equilibrium and obtain basal signals, then quickly withdrawn from the reader, compounds were added to the cells (10 μL of 10× stock solution) and monitoring was resumed. Metabolic effectors were added to the cells at the following final concentrations: 1 uM FCCP, 10 uM antimycin A, 0.5 uM Valinomycin, 5 mM EGTA, 100 mM KCl. Sample respiration profiles are shown in FIG. 6.

When PC12 cells were loaded with either PTCPTE-CTAT or PTCPTE-CFR9 probe and stimulated with 1 uM FCCP, a 2 us increase in probe lifetime was observed. The response with probes was very similar to that obtained with conventional PtCP-BSA probe loaded by transfection. Even after 2-6 hrs of loading with 10 uM of PTCPTE-CFR9, stable lifetime readings and easily detectable response to stimulation by FCCP were observed. The peptide probes can be loaded as quickly as in 2-4 hrs for certain cell types such as primary cells.

PC12 cells loaded for 16 hrs with the PTCPTE-CFR9 probe were also stimulated with the other well-known drugs: antimycin A (inhibitor of the electron transport chain at complex III), potassium ionophore valinomycin (uncoupler of oxidative phosphorylation) and potassium chloride (membrane depolarizing agent). Antimycin A inhibits the respiration decreasing probe lifetime, whereas potassium chloride and electron transport chain uncouplers usually increase O₂ consumption transiently increasing probe lifetime similar to FCCP. In all cases, the cells produced clearly visible responses, which were in agreement with the mode of action of the effector. When the probe was non-specifically adsorbed on the surface of the wells (i.e. without cells), it produced practically no response (a minor peak was due to temperature fluctuations during the addition of stimulant). These results prove that PTCPTE-CFR9 and related TAT probes can be used in intracellular O₂ sensing applications.

### Example 5

### Cell Viability Assessment

Cell viability was assessed by measuring total cellular ATP in the loaded cells using CellTiter-Glo luminescent kit (Promega). PC12 cells were seeded into 96 well plates at concentration 50,000/well, differentiated for 3 days, loaded with peptide probe at a concentration 10 uM for 21 hrs, then washed. CellTiter-Glo reagent was then added to each well and chemiluminescence was measured on the Victor2 plate reader (Perkin-Elmer). The total luminescence signal of unloaded cells was taken as 100% viability. For the cells loaded with probes, no significant changes in ATP were observed and cell viability was retained at 93-95%.

### Example 6

### Oxygen Calibrations for the Intracellular Probes

PC12 cells in a 96-well plate were loaded with the 10 uM of PTCPTE-CFR9 probe for 16 hrs as described in previous examples. The plate with loaded cells was then placed in the hypoxia chamber (Coy Scientific, USA) equilibrated at different pO₂ levels ranging from normoxia (20.8%) to deep hypoxia (1%). To block cellular respiration, 10 uM antimycin A was added to some wells with cells. The plate was then measured on a TR-F plate reader Victor2 (also placed in the hypoxia chamber) at 37° C., taking readings in each well every 2.5 min for about 1-2 hours. After the samples reached temperature and gas equilibrium and stable lifetime signals were produced, these lifetime values were taken for calibration. To obtain lifetime signals at zero O₂, 100 mM of D-(+)-Glucose and 100 ug/ml of glucose oxidase enzyme were added to the wells with cells and lifetime signals were monitored as above For the non-respiring PC12 cells (with antimycin A added), average lifetime under normoxia was 33±2 us, and increased to 65 us in deoxygenated conditions. The respiring cells showed a significant deoxygenation of the monolayer, which was dependent on the external pO₂ in the chamber. The whole calibration shown in FIG. 5 looks very similar to the calibration of the conventional PtCP-BSA probe. Such O2-sensing behaviour of the new probe is well suited for measurement cellular O2 over the whole physiological range 0-21%.

The invention is not limited to the embodiments hereinbefore described which may be varied in construction and detail without departing from the spirit of the invention.

1-36. (canceled)

37. A cell-permeable phosphorescent compound of general formula I, 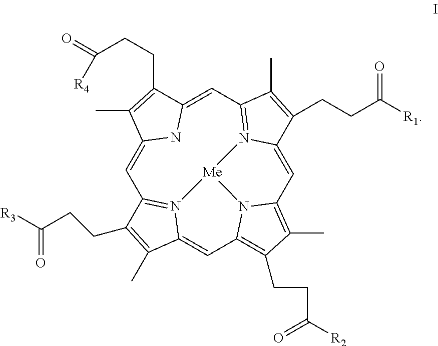
or phosphorescent analogs thereof, wherein:
one of R1 to R4 has a formula X—Y, wherein Y is a cell penetrating peptide, and X is absent or is a chemical linker;
each of the remaining R1 to R4 groups are, independently, uncharged C1-C4 alkoxy groups; and
Me is Pt²⁺ or Pd²⁺,
or
each of R1 to R4 has a formula X—Y, wherein Y is a short cationic peptide comprising at least two arginine residues, and X is absent or is a chemical linker, and wherein the four cationic peptides together provide cell penetrating capability to the compound; and
Me is Pt²⁺ or Pd²⁺.
which probe is capable of measurement of molecular oxygen within live respiring cells by quenched-phosphorescence detection.

38. A compound as claimed in claim 37 in which the uncharged C1-C4 alkoxy group is a methoxy or ethoxy group.

39. A compound as claimed in claim 38 having the general formula II:

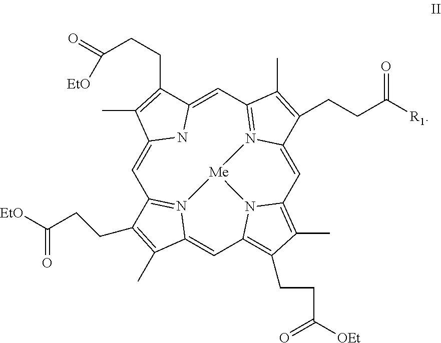

40. A compound as claimed in claim 37, in which Y is a cell penetrating peptide sequence selected from the group consisting of CFRRRRRRRRRR, FRRRRRRRRR, GPRPLPFPRPG, CFGRKKRRQRRR, and a functional variant thereof.

41. A compound as claimed in claim 37, in which the at least one of R1 to R4 is X—Y, and wherein X is selected from the group of common linker structures based on maleimide, pentafluorophenyl, N-succinimide, or an isothiocyanatophenyl moiety.

42. A compound as claimed in claim 41 in which X—Y is maleimide-Y or PEG-maleimide-Y, and in which Y includes a cysteine residue, and wherein the linker is conjugated to Y via a thiol linkage to the cysteine residue.

43. A compound as claimed in claim 42 having a chemical structure selected from the group consisting of:

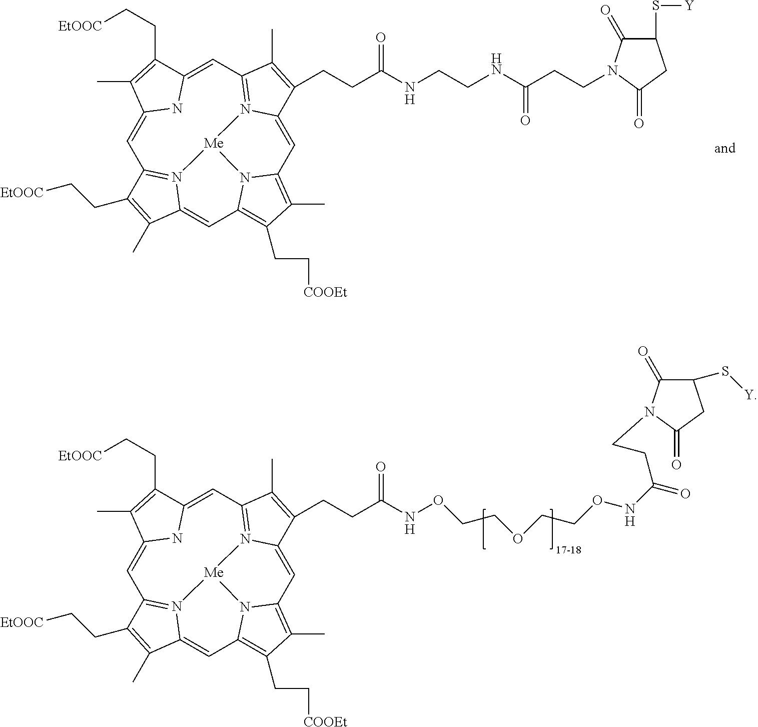

44. A compound according to claim 43 having a chemical structure selected from the group consisting of:

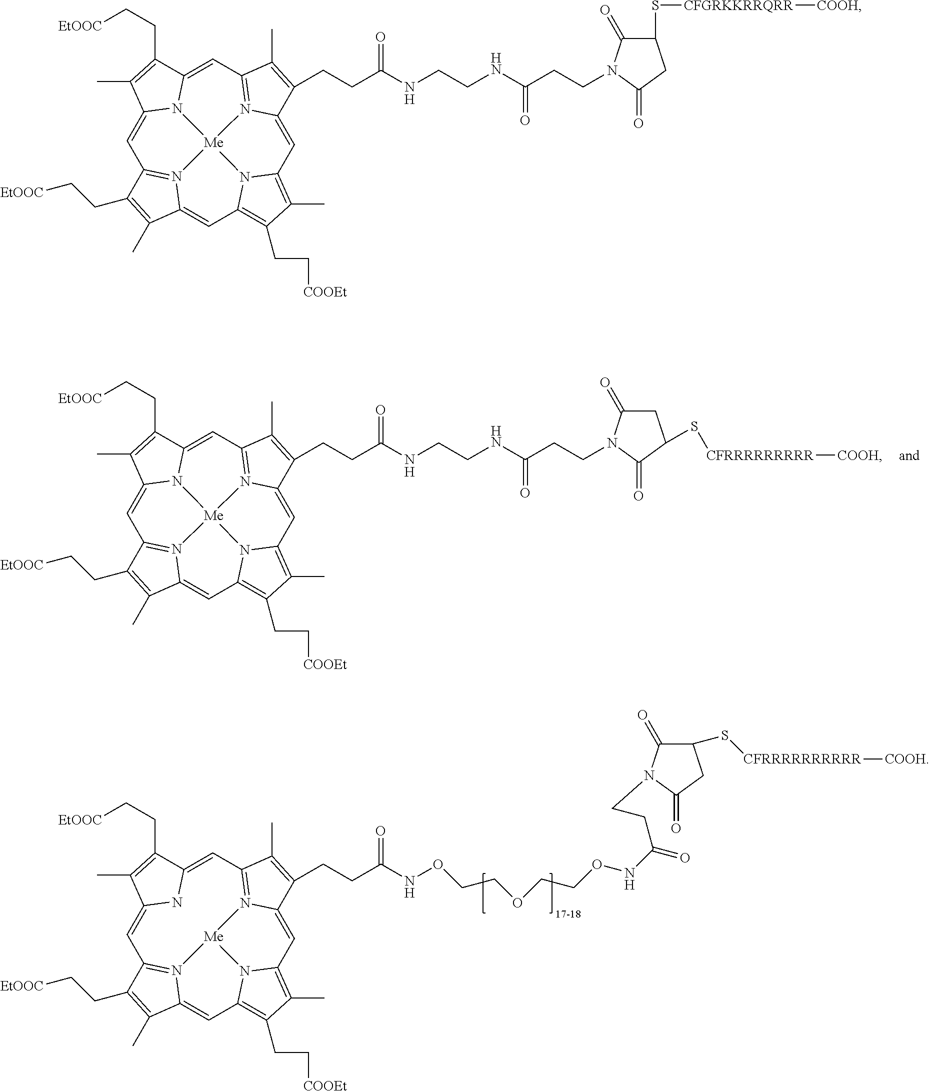

45. A compound according to claim 37 having a chemical structure selected from the group consisting of:

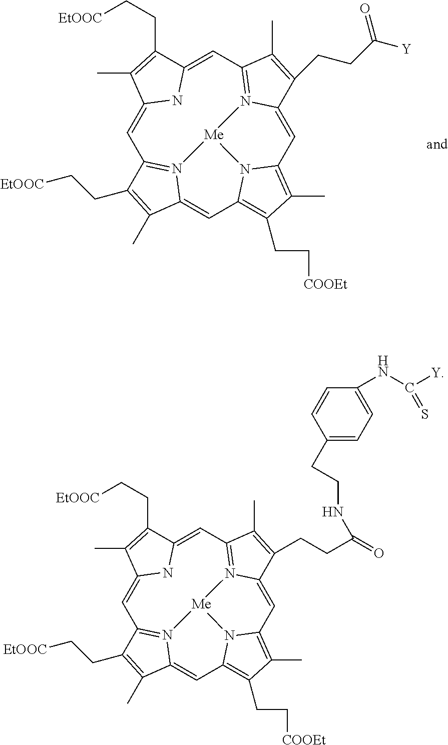

46. A compound according to claim 37 having a chemical structure selected from the group consisting of:

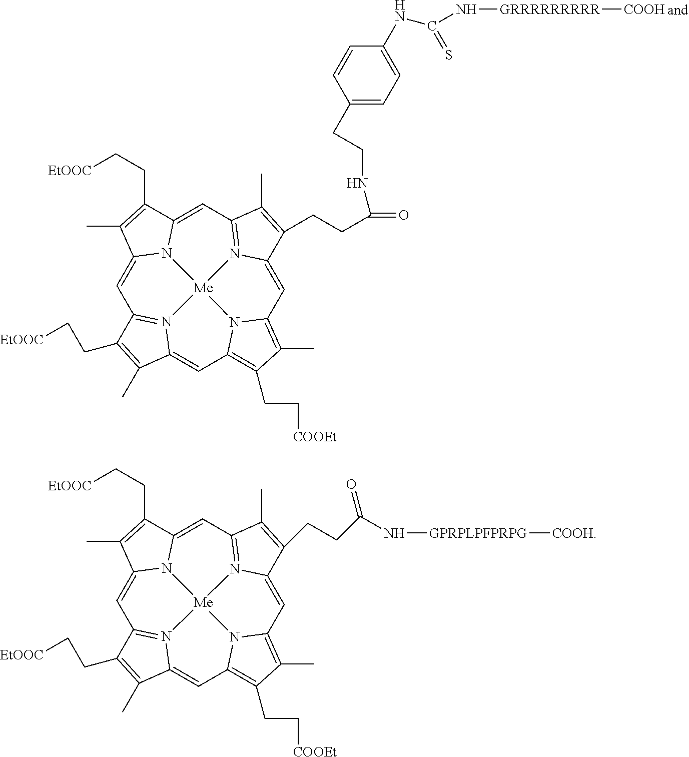

47. A compound as claimed in claim 37 in which the peptide sequence Y is capable of targeting the probe to a specific location within the cell, optionally selected from mitochondria, late endosomes, lysosomes, endoplasmic reticulum or nuclei.

48. A compound as claimed in claim 37 in which the short cationic peptide is di-arginine amidated at a C-terminus and linked via its N-terminus.

49. A compound according to claim 48 having a chemical structure selected from the group consisting of:

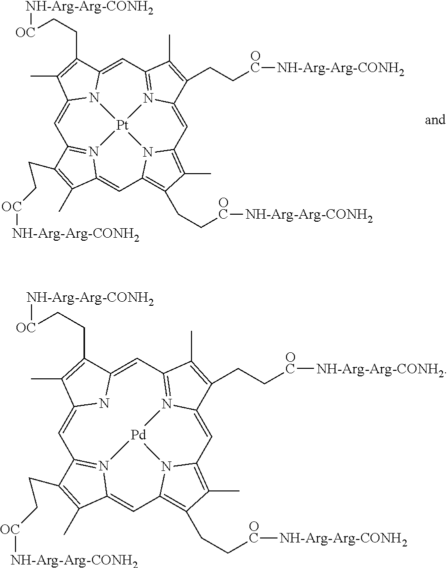

50. A compound as claimed in claim 37, in which the phosphorescent analogs of the compound of general formula I are selected from the group consisting of coproporphyrin III, coproporphyrin-1-ketone, tetra(p-carboxyphenyl)porphine, and a closely related tetrapyrrolic structure.

51. A compound as claimed in claim 50, in which the phosphorescent analogs are selected from the group consisting of: [chemical structure]
